(19) United States
(12) Patent Application Publication
(10) Pub. No.: US 2018/0036348 A1
(43) Pub. Date: Feb. 8, 2018

(54) INTRATHECAL ADMINISTRATION OF MESENCHYMAL STEM CELLS AND DERIVATIVES THEREOF FOR TREATMENT OF PAIN

(71) Applicant: Aidan Research & Consulting LLC, Farmers Branch, TX (US)

(72) Inventor: Neil RIORDAN, I, Southlake, TX (US)

(73) Assignee: Aidan Research & Consulting LLC, Farmers Branch, TX (US)

(21) Appl. No.: 15/669,694

(22) Filed: Aug. 4, 2017

Related U.S. Application Data
(60) Provisional application No. 62/370,706, filed on Aug. 4, 2016.

(51) Int. Cl. A61K 35/28; A61K 9/00

(52) U.S. Cl. CPC A61K 35/28; A61K 9/0085; A61K 9/0019

(57) ABSTRACT

The invention discloses means of treating pain through administration of mesenchymal stem cells or derivatives thereof via an intrathecal manner at a concentration and frequency sufficient to reduce pain. In one embodiment of the invention, treatment of discogenic pain is provided by administration of mesenchymal stem cells intrathecally that are derived from the Wharton's Jelly. In another embodiment the invention teaches administration of supernatants from mesenchymal stem cells, in one embodiment said supernatants comprising of concentrated microvesicles.

### CROSS-REFERENCE TO RELATED APPLICATIONS

This application claims priority to U.S. Provisional Application No. 62/370,706, filed Aug. 4, 2016, which is incorporated herein by reference in its entirety.

### FIELD OF THE INVENTION

The invention pertains to the field of pain management, more specifically, the invention pertains to the field of utilizing stem cells to ameliorate pain by means of administering mesenchymal stem cells. More specifically, the invention teaches the use of intrathecally administered mesenchymal stem cells to reduce pain, in particular discogenic pain in the back of a patient.

### BACKGROUND OF THE INVENTION

Lower back pain (LBP), causes significant impact personally and socially, affecting 70 to 80% of adults least once in a lifetime [1]. In 1998, about 26.3 billion dollars were expended due to LBP in United States [2]. Back pain (BP) is generally self-limited and positive and patients undergoing acute BP may usually become better in one month or can return to work [3, 4]. However, 2 to 7% of the patients show the progress into chronic BP and chronic or regenerative BP may cause 75 to 85% of absence from works [5-7]. Accordingly, in case of occurring acute BP, it is important to apply a method of treating while minimizing side effects to thus relieve pains, which in turn improves functionality, reduces a rate of absence and suppress the progress into chronic BP. Treatment of LBP generally includes prescription of an analgesic agent, for example, acetaminophen, or non-steroid anti-inflammatory drugs (NSAIDs) and also encouraging a patient to continuously retain daily activity [8, 9]. NSAIDs are effective for curing BP a short period of time and, in an aspect of relieving pains, more superior over acetaminophen [10]. In most general, intramuscular application of diclofenac is a method of curing acute pains however, using NSAID often causes side effects in the stomach (and intestines) [11]. There is currently an increasing concern about safety of cyclooxygenase-2 selective NSAIDs for cardiovascular diseases, in particular, thrombotic diseases such as acute myocardial infarction, unstable angina pectoris, cardiac arrest, sudden (cardiac) death [12].

### DESCRIPTION OF THE INVENTION

The invention teaches means of reducing various types of pain by intrathecal administration of mesenchymal stem cells. In a preferred embodiment, the invention teaches that mesenchymal stem cells, or derivatives thereof, possess ability to inhibit discogenic pain when administered in a manner allowing said stem cells or derivatives thereof to access directly into the cerebral spinal fluid. Means of introducing stem cells, or derivatives thereof into said cerebral spinal fluid include intrathecal injections, intraventricular injections and use of specialized delivery devices such as an ommya reservoir.

Various definitions of pain in general are provided for the practice of the invention. In a broad definition, pain is an unpleasant feeling that is different from touch, pressure, heat and cold. Terms such as sharp pain, dull pain, aching pain, stabbing pain, cutting pain or burning pain are often used by patients to describe pain. Generally, the pain induced by nerve injury and showing hyperalgesia is referred as “neuropathic” pain, and the pain caused from the stimulating nociceptor is referred as “nociceptive” pain. For the practice of the current invention, intrathecal administration of mesenchymal stem cells is performed for both types of pain. In a preferred embodiment, mesenchymal stem cells, or products thereof, such as supernatant, purified proteins from supernatants, microvesicles, or exosomes are administered intrathecally for reducing pain generating in the lumbar back area.

Further classifications of pain are described in more detail. For example, clinically, pain can be divided into two classes: acute pain and chronic pain. Acute pain is induced by the damage of skin, body structure or internal organs and/or noxious stimulation caused by diseases, or by the abnormal function of muscle or organ that does not generate actual tissue injury. Chronic pain can be defined as one kind of pain that lasts a period of time that exceeds the common course or healing time of acute diseases, or that is associated with the chronic pathological processes that cause continuous pain, or that relapses for several months or years with certain interval. If pain still exists after treatment that should cure the disease, such pain can be regarded as chronic pain. For the purpose of this invention, chronic pain can be chronic non-palliative or recurrent. The differences between the definition of acute pain and chronic pain are not only semantic differences, but also with important clinic correlation. For example, if acute pain cannot be well-controlled, it can develop to chronic pain. Acute pain is different from chronic pain in the aspects of etiological mechanism, pathology and diagnoses and treatment. In contrast to the transiency of acute pain, chronic pain is induced by the chronic pathological processes in body structure and internal organs, or by peripheral or central nervous system or the extended and sometimes permanent dysfunction thereof. In addition, chronic pain is sometimes attributed to psychological mechanisms and/or environmental factors. Generally, acute pain is non-neuropathic pain and includes common diseases such as arthritis pain, musculoskeletal pain, postoperative pain and fibromyalgia. The majority of such pain is thought to be caused by soft tissue and bone injury, for example arthritis pain, musculoskeletal pain and postoperative pain, and causes inflammatory reactions in normally functional nervous system, with pain just the consequence of inflammatory process. Chronic pain includes neuropathic pain, inflammatory pain and cancer pain, which is associated with hyperalgesia and/or allodynia, wherein hyperalgesia means elevated sensitivity to typical noxious stimulation while allodynia means elevated sensitivity to typical non-noxious stimulation. Somatogenic pain is caused by peripheral sensory nerve injury or infection, including but not limiting to pain caused by peripheral nerve injury, herpesvirus infection, diabetes, causalgia, blood vessel or plexus avulsion, neuralgia, amputation and nodular vasculitis. Neuropathic pain can also be induced by the nerve injury caused by chronic environmental poisoning, infection of human immunodeficiency virus, hypothyroidism, uremia or vitamin deficiency. Its clinical manifestation include but not limit to inflammatory pain, osteoarthritis pain, trigeminal neuralgia, cancer pain, diabetic neuropathy, restless legs syndrome, postherpetic neuralgia, causalgia, brachial vessel plexus avulsion, occipital neuralgia, gout, phantom limb, burn and other forms of neuralgia, neurological and spontaneous pain syndrome. Neuropathic pain is generally considered as chronic pain caused by peripheral or central nervous system injury or diseases. The diseases related to neuropathic pain include long-term peripheral or central neuron sensitization, central sensitization associated with nervous system inhibitory and/or exciting function injury as well as abnormal interaction between parasympathetic and sympathetic nervous system. Many clinical symptoms are related with neuropathic pain or form the basis of neuropathic pain, including for example diabetes, postoperative pain of amputation, lower back pain, cancer, chemical injury or toxin, other serious surgeries, peripheral nerve injury caused by traumatic injury compression, nutrition deficiency, infection such as herpes zoster and HIV. There are numerous causes of pain for which the current invention is useful in addressing and one of skill in the art may utilize basic experimentation to identify optimum dosages and treatment regiments. The invention may be used in the treatment of pain diseases, which include (but not limit to): headache, facial pain, neck pain, shoulder pain, back pain, thoracic pain, abdominal pain, dorsopathy, waist pain, lower limb pain, muscle and bone pain, body pain, vascular pain, gout, arthritis pain, somatoform disorder associated pain, visceral pain, the pain caused by infectious diseases such as AIDS and postherpetic neuralgia, pain associated with multiple bone pain, sickle cell anemia, autoimmune disease, multiple sclerosis or inflammation acute or chronic inflammatory pain, cancer pain, neuropathic pain, injury or surgery caused pain, cancer pain, nociceptive pain, diabetes, peripheral neuropathies, postherpetic neuralgia, trigeminal neuralgia, waist or cervix radiculopathy, glossopharyngeal neuralgia, autonomic nerve reflex pain, reflex sympathetic dystrophy, nerve root avulsion, cancer, chemical injury, toxin, nutrition deficiency, virus or bacteria infection, degenerative osteoarthropathy or the combination thereof.

Specifically, the invention teaches use of intrathecally administered mesenchymal stem cells in reduction and/or amerlioration of back pain, or other types of pain. Conditions often causing pains in low back (that is, the lumbar) and pelvic area of a person are as follows, and methods for examination, diagnosis and/or treatment of these diseases are well known in the art and are presented to allow the practitioner of the invention to perform the teachings of the invention. Herniated Intervertebral Disc (HIVD)—Lumbar Radiculopathy. This condition is often called sciatica and accompanies neurotic disturbance (or disorder) in association with lower limbs to a certain extent. This condition may occur by stimulating the fifth lumbar nerve and the first sacral nerve due to extrusion of nucleus pulposus and, optionally, caused by direct nerve root compression and/or chemical stimulation of substances in the nucleus pulposus. Prevalence of HIVD is about 2% of total population and 10 to 25% among them with HIVD show continued symptoms of six weeks or longer. Cases requiring surgery may range from 5 to 10% of total disc patients. This condition generally expresses serious (acute) symptoms and is often accompanied with low back pain. Some patients say sometimes that previously suffered pain disappeared after having leg pain. The pain becomes increased during sitting, coughing or sneezing. It may be difficult to posture for convenience while reducing pain. When taking a pose bending knees and putting the same to the chest, the disc is expanded and the patient should bend as much as possible to increase an inner radius of a spinal cavity, thus helping easy the pain. There are typically pains from the hip toward posterior or postero-lateral parts up to the ankle or foot. For a neuromuscular disease at the center of lumbar (L1-L3), referred pain is expressed at anterior thigh but, usually, not extended below the knee. HIVD on this site is not higher than 5% of overall HIVD. Radiculopathy refers to disease of the spinal nerve roots (from the Latin radix for root). Radiculopathy produces pain, numbness, or weakness radiating from the spine. Radiculopathies are categorized according to which part of the spinal cord is affected. Thus, there are cervical (neck), thoracic (middle back), and lumbar (lower back) radiculopathies. Lumbar radiculopathy is also known a sciatica. Radiculopathies may be further categorized by what vertebrae they are associated with. For example, radiculopathy of the nerve roots at the level of the seventh cervical vertebra is termed C7 radiculopathy; at the level of the fifth cervical vertebra, C5 radiculopathy; at the level of the first thoracic vertebra, T1 radiculopathy. Spinal Stenosis. This refers to a condition wherein, owing to certain reasons, a spinal canal, nerve root canal or intervertebral cavity is narrowed to induce low back pain or cause a variety of complicated neuroses on legs. In general, the nucleus pulposus and fibrous ring begin to be degenerated after 30 years old, and thus, a part of intervertebral disc adhered to the spin is detached to remain bone spur. Simultaneously, posterior joint protrusion, vertebral arch, ligament flavum, may also be deformed and thickened to make all area around the spinal canal to be narrowed. Further, the spin is bent in front and rear directions to directly press the spinal cord and nerve roots and cause blood flow disorder, resulting in occurrence of symptoms. Low back pain is very often expressed, dislike lumbar intervertebral herniation, sensory disorder and weakened muscular strength as well as sharp, squeezing or burning pain arises around the hip and anus accompanied with sensory disorder and weakened muscular strength. Such symptoms as described above are generally worse under cold weather or during exercising but improved under warm weather or when a patient is at ease. For symptoms often arising and becoming serious, these symptoms disappear when the patient bends at the waist or stops walking but crouches down to rest, while being repeated by walking again. Such a condition as described above typically refers to as neurogenic intermittent claudication. As an extent of stenosis is increased, a walking distance may be shortened. Conditions of a sensory disorder, such as sensibility loss or numbness, may be expressed over a wide area of the body along with calf, ankle, knee, thigh, hip and inguinal regions. Anus dysfunction is the latest symptom. Degenerative Disc Disease: This refers to a disc condition with degraded mechanical and/or chemical properties of the intervertebral disc due to various causes including, i.e., ageing, trauma, high impacting activity, type of works, smoking, genetic factors, and so forth. A mechanical disorder having low back pain during bending or stretching the body and neurotic disorders having leg pain during sitting down or walking are expressed. A degenerative intervertebral disc disease often arises without specific symptoms. Patients with typical degenerative disc explain that a pain arises when getting up in the morning, however, disappears during walking about 1 hour. At a molecular level, disc degeneration associated with the aging process is generally associated with the loss of proteoglycan from the nucleus pulposus of the spinal discs and a reduction of the disc's ability to absorb shock between vertebrae. Although some affected patients may not exhibit symptoms, many affected patients suffer from chronic back or neck pain in addition to arm and/or leg pain. Pain associated with disc degeneration may become debilitating and may greatly reduce a patient's quality of life. In some embodiments, the degenerative disc disease can include an intervertebral disc herniation. As used herein “intervertebral disc herniation” includes local displacement of disc material beyond the limits of the intervertebral disc space. The disc material may be nucleus pulposus, cartilage, fragmented apophysical bone, annular tissue or any combination thereof. Displacement of disc material may put pressure on the exiting spinal nerve and/or cause an inflammatory reaction leading to radiculopathy, weakness, numbness, and/or tingling in the arms or legs. In general, most degenerative disc disease takes place in the lumbar area of the spine. Lumbar disc herniation occurs 15 times more often than cervical disc herniation, and it is one of the most common causes of lower back pain. The cervical discs are affected 8% of the time and the upper-to-mid-back (thoracic) discs only 1-2% of the time. Sometimes degenerative disc disease can lead to compression of the nerve roots of the spine resulting in very painful neurological symptoms. Nerve roots (large nerves that branch out from the spinal cord) may become compressed resulting in neurological symptoms, such as sensory or motor changes. For example herniation of the nucleus pulposus often is accompanied by lower back pain that worsens in the sitting position and pain that radiates to the lower extremities. The radiating pain, for example, in sciatica is often described as dull, burning or sharp pain, accompanied by intermittent sharp electric shock sensation, numbness, and tingling, motor or sensory defects of the respective nerve root and/or reflex abnormalities. Spobdylolisthesis: In this condition the vertebra comprises multiple small bonds stacked in a tower form. Joint protrusions in a ring type ring placed at a rear part of the vertebra fix upper and lower bones. Spobdylolisthesis refers to the wherein the upper spinal bone slides and is forced out toward the front due to various causes such as damage of joint protrusions. Major causes may include degeneration of discs and joints, congenital spinal abnormality, accident, impact-derived fracture of spinal joint protrusion, and the like. In a case of standing up after sitting down or stretching (or bending) backward at the waist, low back pain is caused. When getting up in the morning, low back pain is caused. In a case of taking a stand for a long time or walking a long distance, it causes pains in the waist, hip and/or below knees. By passing a hand over the spine during straightening the waist body and touching the body, depressed parts are found. Walking with faltering steps like a duck is found. Facet Joint Syndrome: In this condition pain is generated through nerves distributed over a facet joint since a joint membrane of the facet joint sensitive to pain has acute trauma or degenerative modification, thus causing fracture of the face joint membrane or arthritis. Pain of which the position is not certainly detected or traced. The patient possesses symptom of strain from the hip to the posterior thigh (similar to intervertebral disc disorder). Radiating pain of the lower limbs is not usually broadened below the knees. Pain increasing when getting up in the morning, however, decreasing during activity. Pain decreasing during bending frontward, however, increasing during stretching and bending in lateral sides. Intervertebral disc herniation includes a rupture of the annulus fibrosis, through which the inner disc material (nucleus pulposus) extrudes, protrudes, bulges, migrates and/or re-herniates. Sometimes disc extrusions may be displaced so much that it has lost continuity with the parent disc. When this happens the extrusion is called sequestration. Thus, the methods of the present application can be used to treat pain associated with ruptures, protrusions, bulges, extrusions, re-herniation, and migration, fragmented, and/or sequestrated nucleus pulposus.

Some definitions are provided below to clarify concepts disclosed in the specification and to help one of skill in the art in the practice of the invention:

“acute pain” is defined as the pain caused by the injury of skin, body structure or internal organs and/or noxious stimulation of the diseases, or the pain caused by the abnormal function of muscle or internal organs that does not produce real tissue injury.

“chronic pain” is defined as the pain that lasts a period of time that exceeds the common course or healing time of acute diseases, or that is associated with the chronic pathological processes that cause continuous pain, or that relapses for several months or years with certain interval. If pain still exists after treatment that should cure the disease, such pain can be regarded as chronic pain. The time duration that the pain lasts depends on the nature of pain and the treatment process associated with pain. If the pain exceeds common treatment process, then this pain is chronic. Chronic pain includes but not limits to headache, facial pain, neck pain, shoulder pain, thoracic pain, abdominal pain, back pain, waist pain, lower limb pain, muscle and bone pain, somatoform disorder associated pain, visceral pain, painful diabetic neuropathy, vascular pain, gout, arthritis pain, cancer pain, autonomic nerve reflex pain, the pain caused by infectious diseases such as AIDS and herpes zoster, the pain caused by autoimmune disease such as rheumatism, the pain caused by acute or chronic inflammation, postoperative pain and post-burning pain. The intrathecal administration of mesenchymal stem cells disclosed by this invention can efficiently treat the chronic pain defined as above, and the drugs disclosed by this invention can be used to treat hyperalgia accompanied with other diseases, including hyperalgesia, allodynia, algesia enhancement and pain memory enhancement. This invention will improve the treatment of pain.

“headache” can be divided into primary headache and secondary headache. Primary headache includes tension headache, migraine headache and cluster headache, and secondary headache is caused by other diseases. Headache is caused when pain sensitive tissue on head and face undergo lesion or get stimulated. These pain sensitive tissues are distributed on scalp, face, oral cavity and throat. They are mainly muscles and vessels in head with abundant nerve fibers and sensitive to pain, therefore headache is caused when these tissues are injured.

“facial pain” includes but not limits to trigeminal neuralgia, atypical facial pain, facial palsy and facial spasm.

“trigeminal neuralgia” is a unique chronic painful disease, also referred as tic douloureux, representing transient, paroxysmal and repeated electric shock-like severe pain in trigeminal nerve area, or accompanied with ipsilateral facial spasm. Trigeminal neuralgia can be divided into two classes: primary and secondary. Primary trigeminal neuralgia means no neurological sign is found clinically and no organic disease is detected. Secondary trigeminal neuralgia means neurological signs are found clinically and organic diseases such as tumor and inflammation are detected.

“atypical facial pain” represents pain caused by various diseases, appearing as burning pain, non-intermittent and independent of particular action or stimulation. The pain is often bilateral and exceeds the area of trigeminal nerve to even cervical skin. The etiology can be the stimulation of nasosinusitis, malignant tumor, jaw and skull base infection or injured trigeminal nerve.

“neck pain, back pain, shoulder pain” represent the pain caused by acute or chronic muscle strain and bone joint degeneration and injury. The common diseases that cause neck, shoulder and upper limb pain include cervicoshoulder myofascitis, neck desmitis, cervical spondylopathy, scapulohumeral periarthritis, thoracic outlet syndrome, external humeral epicondylitis, etc. Alternatively, these terms represent the pain cause by autoimmune diseases rheumatoid arthritis, ankylosing spondylitis and rheumatic arthritis. Other diseases that can cause neck pain, back pain and shoulder pain are tumors on neck and shoulder, neuritis, arteriovenous disease and various infections as well as referred pain induced by lesions of thoracic and abdominal organs.

“thoracic, abdominal, and back pain” represent the pain caused by diseases in thoracic and abdominal organs and thoracic and abdominal wall tissues, including but not limiting to intercostal neuralgia, intercostal chondritis, angina pectoris, abdominal pain (acute abdominal organ pain) and waist and back myofascial pain syndrome.

“waist pain, lower limb pain” represent low back, lumbosacral, sacroiliac, hip, buttocks and lower limb pain. Generally, waist and lower limb pain is not independent disease, but the common feature of various diseases, with diverse clinical manifestation and complex etiology. Such pain is mainly induced by degeneration and injury, including but not limiting to the pain involving lumbar disc herniation, acute lumbar sprain, ischialgia, osteoporosis, third lumbar trans-verse process syndrome, piriformis syndrome, knee osteoarthritis, coccygodynia and calcanodynia.

“muscle and bone pain” includes but not limits to myofascial pain, trauma-caused pain and chronic regional pain syndrome.

“painful diabetes” represents the pain caused by nerve injury concurrent with diabetes. The nerve injury in diabetes is caused at least partly by blood flow reduction and hyperglycemia. Some diabetes patients do not suffer neuropathy, while others suffer this disease at early stage. Diabetic neuropathy can be divided into mononeuropathy that involves one or several lesion sites and systemic polyneuropathy. The polyneuropathy can be dispersive and symmetrical, generally and mainly involving mode of sensation (Merrit's Textbook of Neurology, the 9.sup.th version). The manifestation of diabetic neuropathy includes plant nerve dysfunction, and cause dysregulation involving heart, smooth muscle and gland, resulting in hypotension, diarrhea, constipation and impotence. Diabetic neuropathy often develops in stages. The early stage takes place in nerve ending area. Plant neuropathy or sensory neuropathy occurs in feet and brain neuropathy occurs in face and periocular area with intermittent pain and the sense of tingling. In the following stages, the pain become more severe and occurs more frequently. Finally, when analgesia happens in one area, the disease develops into painless neuropathy. Due to lack of pain as the sign of injury, the risk of severe tissue damage is greatly increased.

“visceral pain” includes but not limits to the pain of inflammatory bowel syndrome (IBS), with or without chronic fatigue syndrome (CFS), inflammatory bowel disease (IBD) and interstitial cystitis.

“vascular pain” represents the pain generated by the following one or more factors. Firstly, improper perfusion of tissue, resulting in temporary or continuous ischemia, e.g the ischemia in limb muscles during physical exercise. Secondly, delayed change, e.g. ulcer or gangrene in skin or abdominal organs. Thirdly, the sudden and accelerated change of diameter of great vessels, e.g. the change of arterial aneurysm. Fourthly, aortic rupture, resulting in blood spillover and the stimulation of nociceptive fibers in peritoneum or pleura parietal layers. Fifthly, strong cramp caused by the severe stimulation of artery endothelium by intra-arterial injection. Sixthly, the damage of venous return, leading to a large number of edema of rapidly expanded fascia compartment (Bonica's Management of Pain, Volume 1 (the 2.sup.nd version)). The examples include but not limit to arteriosclerosis obliterans, thromboangiitis angiitis, acute arterial closure, embolism, congenital arteriovenous aneurysm, vasospasm diseases, Rayaud's disease, acrocyanosis, acute venous closure, thrombophlebitis, varicosity and lymphedema.

“cancer pain” represents the pain occurs during the development process of malignant tumor. Currently, it is thought that there are three mechanisms of cancer pain, i.e. the pain caused directly by cancer development, the pain caused after cancer treatment and the concurrent painful diseases of cancer patients.

“autonomic nerve reflex pain” represents the pain caused by “reflex sympathetic dystrophy”. For reflex sympathetic dystrophy, after the body suffers acute or chronic injury, severe ambulatory pain occurs and the body is sensitive to the sense of touch and pain, probably accompanied with edema and blood disorder, following symptoms like skin and musculoskeletal nutrition dystrophia and atrophy.

“postoperative pain” represents a complex physiological response of body to the disease itself and the tissue injury caused by operation, showing an unpleasant psychological and behavior experience.

“arthritis pain” includes but not limits to the pain caused by osteoarthritis, rheumatoid arthritis, joint ankylosing spondylitis, psoriatic arthropathy, gout, pseudo gout, infectious arthritis, tendinitis, bursitis, bone damage and joint soft tissue inflammation.

“postherpetic neuralgia” represents the subcutaneously long-standing severe pain in rash site after the healing of the rash of herpes zoster.

“nociceptive pain” represents the pain caused by the tissue injury delivered by nociceptors, or the pain caused by the extended excitement of nociceptors. The pain caused by the extended excitement of nociceptors can be induced by both the persisting noxious stimulation of nociceptors and the sensitization thereof, or they can be induced by these factors and extended by their persistence, various reflex mechanisms and other factors.

“algesia” represents the neuromechanism for detecting noxious stimulation. Algesia involves two steps: the transduction of noxious stimulation by peripheral nerve ending and delivering these signals to central nervous system.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSCcan be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC , Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In one embodiment MSC donor lots are generated from umbilical cord tissue. Means of generating umbilical cord tissue MSC have been previously published and are incorporated by reference [13-19]. The term “umbilical tissue derived cells (UTC)” refers, for example, to cells as described in U.S. Pat. No. 7,510,873, U.S. Pat. No. 7,413,734, U.S. Pat. No. 7,524,489, and U.S. Pat. No. 7,560,276. The UTC can be of any mammalian origin e.g. human, rat, primate, porcine and the like.

In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue for administration to allow direct contact with cerebral spinal fluid in a manner to reduce pain are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes.

Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIB ERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells. Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

Means of intrathecal administration of mesenchymal stem cells are known in the art. The following descriptions are provided to guide one of skill in the art to practice the invention. In a preferred embodiment patients with pain in general, and preferably patients with back pain, and more preferably patients with discogenic back pain are administered with intrathecal doses of stem cells. The references and procedures mentioned are incorporated by reference. Hammadi et al., administered a mean total mononuclear cell count per product of 5×10⁸ (range 1˜8) G-CSF mobilized cells in 50 patients suffering from multiple sclerosis intrathecally. The rationale was that mobilization would increase mesenchymal stem cell counts in peripheral blood. spinal injection of the cell product intrathecally through the fifth lumbar vertebrae in sitting position under local anesthesia by lidocaine within 24 hours of collection. The procedure was repeated 1˜8 times (mean 2.14) within 6˜8 weeks. The period of follow up is 12 months. A subjective clinical improvement was referred by 42 patients soon after 2 weeks of starting therapy (84%), while an objective reduction in the EDSS score of more than 1 (from 1 to 1.5) was observed in 24 patients (48%). No major changes of MR imaging was seen. However there was no increase of Gadolinium enhancement in 40 patients during the period of follow up. This cell therapy did not prevent relapses in 8 cases, but they were milder than usual, and quickly controlled by cortisone treatment. The only complication in 90% of cases was transient backache and meningism. No serious side effects where noted [20]. Kishk et al reported that in contrast to our findings of intrathecal MSC preventing pain, the opposite was observed. They treated 44 spinal cord injury patients with monthly intrathecal autologous mesenchymal stem cells for 6 months and 20 subjects, who would not agree to the procedures, served as controls. All subjects received rehabilitation therapies 3 times weekly. Subjects were evaluated at entry and at 12 months after completing the 6-months intervention. By the ASIA Impairment Scale, ASIA grading of completeness of injury, Ashworth Spasticity Scale, Functional Ambulation Classification, and bladder and bowel control questionnaire. No differences were found in baseline measures and descriptors between the MSC group and control group. Although a higher percentage of the MSC group increased motor scores by 1-2 points and changed from ASIA A to B, no significant between-group improvements were found in clinical measures. Adverse effects of cells included spasticity and, in 24 out of the 43 patients developed neuropathic pain [21]. Yamout et al. performed intrathecal injection of ex-vivo expanded autologous bone marrow derived mesenchymal stem cells in 10 patients with advanced multiple sclerosis. Patients were assessed at 3, 6 and 12 months. Assessment at 3-6 months revealed EDSS improvement in 5/7, stabilization in 1/7, and worsening in 1/7 patients. MRI at 3 months revealed new or enlarging lesions in 5/7 and Gadolinium (Gd+) enhancing lesions in 3/7 patients. Vision and low contrast sensitivity testing at 3 months showed improvement in 5/6 and worsening in 1/6 patients. No serious adverse effects where noted [22]. Karussis et al. reported 15 patients with MS and 19 with ALS who were treated with a mean of 63.2×10(6) (2.5×10(6)) MSCs was intrathecally (n=34) and intravenously (n=14). Twenty-one patients had injection-related adverse effects consisting of transient fever, and 15 reported headache. No major adverse effects were reported during follow-up. The mean ALSFRS score remained stable during the first 6 months of observation, whereas the mean (SD) EDSS score improved from 6.7 (1.0) to 5.9 (1.6). Magnetic resonance imaging visualized the MSCs in the occipital horns of the ventricles, indicating the possible migration of ferumoxides-labeled cells in the meninges, subarachnoid space, and spinal cord. Immunological analysis revealed an increase in the proportion of CD4(+)CD25(+) regulatory T cells, a decrease in the proliferative responses of lymphocytes, and the expression of CD40(+), CD83(+), CD86(+), and HLA-DR on myeloid dendritic cells at 24 hours after MSC transplantation [23]. Dongmei et al. 14 cases of SCA and 10 cases of MSA-C were given UC-MSC by weekly intrathecal injection, at a dose of 1×10(6)/kg four times as one course. All the patients received one course of treatment, except three patients who received two courses. The movement ability and quality of daily life were evaluated with the International Cooperative Ataxia Rating Scale (ICARS) and Activity of Daily Living Scale (ADL) and the scores compared with those before cell therapy. A follow-up of 6-15 months was carried out for all of the patients. The results showed that the ICARS and ADL scores were significantly decreased 1 month after treatment (P<0.01). The symptoms, including unstable walking and standing, slow movement, fine motor disorders of the upper limbs, writing difficulties and dysarthria, were greatly improved except for one patient, who had no response. The observed side-effects included dizziness (four patients), back pain (two cases) and headache (one case), which disappeared within 1-3 days. During the follow-up, 10 cases remained stable for half a year or longer, while 14 cases had regressed to the status prior to the treatment within 1-14 months (an average of 3 months) [24]. Kim et al reported 37 patients with ALS who received autologous MSCs via intrathecal injection in two monthly doses. After a 6-month follow-up period, the patients were categorized as responders and non-responders based on their scores on the revised ALS Functional Rating Scale (ALSFRS-R). Biological markers including β-fibroblast growth factor-2, stromal cell-derived factor-1α, vascular endothelial growth factor (VEGF), insulin-like growth factor-1, brain-derived neurotrophic factor, angiogenin (ANG), interleukin (IL)-4, IL-10, and transforming growth factor-β(TGF-β) were measured in the MSC cultures and their levels were compared between the responders and nonresponders. To confirm the markers' predictive ability, MSCs isolated from one patient in each group were transplanted into the cisterna magna of mutant SOD1(G93A) transgenic mice to measure their lifespans, locomotor activity, and motor neuron numbers. The levels of VEGF, ANG, and TGF-β were significantly higher in responders than in nonresponders [25]. Miao et al. reported on 100 patients who underwent subarachnoid placement of umbilical cord mesenchymal stem cells. Technical difficulties in patients in the form of localization of subarachnoid space, number of attempts, and post-procedural complications were evaluated. Functional evaluation was done using Hauser Ambulation Index (HAI) by the stem cell transplant team on a regular basis. All patients were followed-up for more than 1 yr after the treatment. Clinical symptoms, related biochemical index and photographic examinations were observed regularly. We encountered technical difficulties in 31 patients (31%) in the form of general anesthesia supplementation and difficulty localizing the lumbar space. Side effects (headache, low-grade fever, low back pain and lower limb pain) were observed in 22 (22%) patients, which were treated with symptomatic therapy within 48 h. One year after the treatment, functional indices improved in 47 patients (47%): 12 patients with spinal cord injury, 11 patients with cerebral palsy, 9 patients with post-traumatic brain syndrome, 9 patients with post-brain infarction syndrome, 3 patients with spinocerebellar ataxias, and 3 patients with motor neuron disease. The authors concluded that intrathecal administration of umbilical cord mesenchymal stem cells is a safe and effective way to treat neurological disorders [26].

In some embodiments of the invention, pain, more specifically, back pain, is treated by administration of mesenchymal stem cells via intraventricular injection. In one embodiment intraventricular injection is accomplished via an Ommaya reservoir. Use of Ommaya reservoir for administration of stem cells, and other cellular therapies has been previously reported and is incorporated by reference [27-32].

In one embodiment of the invention mesenchymal stem cells are administered in proximity to a pain marker. While generally the invention teaches intrathecal administration of mesenchymal stem cells for relief of pain, in some embodiments, specific localization of said mesenchymal stem cells is desired in proximity to originating source of pain. Pain marker(s) are considered contributors in various ways to (or otherwise indicative of) the generation or transmission of discogenic pain. One such pain marker relates to the presence of nociceptors. Normally, intervertebral discs are substantially avascular and only sparsely innervated at the outer margins of the disc annulus. These unmyelinated, substance P (SP) or calcitonin gene-related peptide (CGRP) containing fibers are typically unresponsive and termed silent nociceptors. SP and CGRP are believed to be the sensory transmitters of nociceptive information. As degeneration proceeds, nerves can follow microvessels and grow deeper into discs, which may occur for example either peripherally or via the endplate. This nerve and vessel in-growth is facilitated by degeneration-related decreases in disc pressure and proteoglycan content. Thus by administering a diagnostic agent that binds SP or CGRP, the SP and CGRP conjugate will have an abnormally high concentration and, therefore, imaging will show a high signal to noise ratio (e.g., 2/1, 3/1, 4/1, 5/1, 6/1, 7/1, 8/1, 9/1, 10/1 etc.) and the pain generator will be clearly imaged and diagnosed in the area of “hot spots”. This provides a means of localizing administration of mesenchymal stem cells. In some embodiments mesenchymal stem cells are temporarily immobilized by administration using a biodegradable matrix.

In some embodiments, mesenchymal stem cells may be administered at locations that are identified using agents capable of localizing origination of pain in the vertebral column. Diagnostic agents may be labeled in vivo by administering a label that binds with the pain marker. In some embodiments, the neuronal pain marker that can be labeled include, without limitation: TRK-.alpha.; anti-TRK .alpha. antibody; nerve growth factor (NGF); anti-NGF antibody; NGF antagonist; anti-NGF antagonist antibody; PGP 9.5; SYN; peripherin; or other form of nerve antibodies or related materials in general. Other materials such as neurofilament 200 kDa (NF200) may also be the target of such labeling and subsequent imaging. In some embodiments, endogenous substances such as TrkA or NGF may be targeted as the pain markers for labeling, or related antibodies or other substances having particular binding affinity or specificity to such substances may be bound to them in the area of pain and then thereafter provide the binding site for targeted labels to be subsequently delivered. In this regard, it is to be appreciated that various forms of binding agents are broadly contemplated hereunder this description. Nerves usually accompany blood vessels, but can be found as isolated nerves fibers in the disc matrix. These non-vessel-associated fibers found in back pain patients have been observed to express growth-associated protein 43 (GAP43) as well as SP. Small disc neurons contain CGRP and also express the high-affinity nerve growth factor (NGF) receptor, tyrosine kinase A (trkA). Disc inflammation has been observed to cause an increase in CGRP positive neurons. A recent study showed that NGF is expressed in microvascular blood vessels in a painful lumbar disc, and that there are trkA (TRK-.alpha.) expressing nerve fibers adjacent to the vessels that enter painful discs primarily through the endplate. Along with nerves growing into degenerated discs are specialized nerve support cells termed ‘glia’ or Schwann cells localized using glial fibrillary acidic protein (GFAP). Accordingly, various such materials may provide the requisite binding affinity or specificity to painful regions (or highly innervated regions) to play the role as the pain marker for the diagnostic agent to bind to or they may be labeled and then administered to identify the pain marker or the pain generator. In one embodiment, for example, TrkA antibody (or other binding agent) is labeled and delivered for binding and visualization to the pain generator. In another embodiment, NGF itself is labeled and delivered as a diagnostic agent, which binds the pain marker TrkA. Some of these agents are further described in Published PCT Patent Applications are herein incorporated in their entirety by reference thereto: WO 2004/032870; WO 2004/058184; WO 2004/073653; WO 2004/096122; and WO 2005/000194.

Various aspects of the invention of the invention relating to the above are enumerated in the following paragraphs:

Aspect 1. A method of reducing pain in a patient comprising administration of mesenchymal stem cells intrathecally in said patient.

Aspect 2. The method of aspect 1, wherein said pain is selected from a group comprising of: a) neuropathic pain; b) nociceptive pain; c) phantom pain; d) psychogenic pain; e) incident pain; f) breakthrough pain; g) discogenic pain and h) idiopathic pain.

Aspect 3. The method of aspect 1, wherein said pain is chronic or acute.

Aspect 4. The method of aspect 1, wherein said mesenchymal stem cells are administered at a concentration of 100,000 cells to 50 million cells.

Aspect 5. The method of aspect 1, wherein said mesenchymal stem cells are administered at a concentration of 500,000 cells to 10 million cells.

Aspect 6. The method of aspect 1, wherein said mesenchymal stem cells are administered at a concentration of 1 million cells to 5 million cells.

Aspect 7. The method of aspect 1, wherein said mesenchymal stem cells are administered at a concentration of approximately 2 million cells.

Aspect 8. The method of aspect 1, wherein said mesenchymal stem cells are administered in a liquid media that is biocompatible with cerebral spinal fluid.

Aspect 9. The method of aspect 1, wherein said mesenchymal stem cells are selected from a group of cells comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Aspect 10. The method of aspect 9, wherein said mesenchymal stem cells express markers selected from a group comprising of: a) CD73; b) CD90; and c) CD105.

Aspect 11. The method of aspect 9, wherein said mesenchymal stem cells lack expression of markers selected from a group comprising of: a) CD45; b) CD34; and c) CD14.

Aspect 12. The method of aspect 11, wherein said mesenchymal stem cells express CD34.

Aspect 13. The method of aspect 9, wherein said mesenchymal stem cells express CD56.

Aspect 14. The method of aspect 9, wherein said mesenchymal stem cells are capable of adherent growth in a liquid culture.

15. The method of aspect 9, wherein said mesenchymal stem cells are capable of differentiating into one or more lineages selected from: a) adipocytic; b) chondrocytic; and c) osteocytic.

Aspect 16. The method of aspect 9, wherein said mesenchymal stem cells maintain a normal karyotype upon passaging for more than 20 cell doublings.

Aspect 17. The method of aspect 9, wherein said mesenchymal stem cells are capable of producing HGF-1 in vitro.

Aspect 18. The method of aspect 9, wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Aspect 19. The method of aspect 9, wherein said mesenchymal stem cells from umbilical cord tissue do not express markers selected from a group comprising of: a) CD117; b) CD31; c) CD34; and CD45;

Aspect 20. The method of aspect 9, wherein said mesenchymal stem cells from umbilical cord tissue express, relative to a human fibroblast, increased levels of interleukin 8 and reticulon 1

Aspect 21. The method of aspect 9, wherein said mesenchymal stem cells from umbilical cord tissue have the potential to differentiate into cells of at least a skeletal muscle, vascular smooth muscle, pericyte or vascular endothelium phenotype.

Aspect 22. The method of aspect 9, wherein said mesenchymal stem cells from umbilical cord tissue express markers selected from a group comprising of: a) CD10; b) CD13; c) CD44; d) CD73; and e) CD90.

Aspect 23. The method of aspect 9, wherein said umbilical cord tissue mesenchymal stem cell is an isolated umbilical cord tissue cell isolated from umbilical cord tissue substantially free of blood that is capable of self-renewal and expansion in culture,

Aspect 24. The method of aspect 23, wherein said umbilical cord tissue mesenchymal stem cells has the potential to differentiate into cells of other phenotypes.

Aspect 25. The method of aspect 24, wherein said other phenotypes comprise: a) osteocytic; b) adipogenic; and c) chondrogenic differentiation.

Aspect 26. The method of aspect 9, wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Aspect 27. The method of aspect 9, wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Aspect 28. The method of aspect 9, wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1

Aspect 29. The method of aspect 9, wherein said umbilical cord tissue derived cells express markers selected from a group comprising of: a) TRA1-60; b) TRA1-81; c) SSEA3; d) SSEA4; and e) NANOG.

Aspect 30. The method of aspect 9, wherein said umbilical cord tissue-derived cells are positive for alkaline phosphatase staining.

Aspect 31. The method of aspect 9, wherein said umbilical cord tissue-derived cells are capable of differentiating into one or more lineages selected from a group comprising of; a) ectoderm; b) mesoderm, and; c) endoderm.

Aspect 32. The method of aspect 9, wherein said bone marrow derived mesenchymal stem cells possess markers selected from a group comprising of: a) CD73; b) CD90; and c) CD105.

Aspect 33. The method of aspect 9, wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; 1) 6-19; m) thrombomodulin; n) telomerase; o) CD10; p) CD13; and q) integrin beta.

Aspect 34. The method of aspect 9, wherein said bone marrow derived mesenchymal stem cell is a mesenchymal stem cell progenitor cell.

Aspect 35. The method of aspect 34, wherein said mesenchymal progenitor cells are a population of bone marrow mesenchymal stem cells enriched for cells containing STRO-1

Aspect 36. The method of aspect 35, wherein said mesenchymal progenitor cells express both STRO-1 and VCAM-1.

Aspect 37. A method of aspect 35, wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Aspect 38. The method of aspect 9, wherein said bone marrow mesenchymal stem cells lack expression of CD14, CD34, and CD45.

Aspect 39. The method of aspect 37, wherein said STRO-1 expressing cells are positive for a marker selected from a group comprising of: a) VACM-1; b) TKY-1; c) CD146 and; d) STRO-2

Aspect 40. The method of aspect 9, wherein said bone marrow mesenchymal stem cell express markers selected from a group comprising of: a) CD13; b) CD34; c) CD56 and; d) CD117

Aspect 41. The method of aspect 40, wherein said bone marrow mesenchymal stem cells do not express CD10.

Aspect 42. The method of aspect 40, wherein said bone marrow mesenchymal stem cells do not express CD2, CD5, CD14, CD19, CD33, CD45, and DRII.

Aspect 43. The method of aspect 40, wherein said bone marrow mesenchymal stem cells express CD13,CD34, CD56, CD90, CD117 and nestin, and which do not express CD2, CD3, CD10, CD14, CD16, CD31, CD33, CD45 and CD64.

Aspect 44. The method of aspect 9, wherein said skeletal muscle stem cells express markers selected from a group comprising of: a) CD13; b) CD34; c) CD56 and; d) CD117

Aspect 45. The method of aspect 44, wherein said skeletal muscle mesenchymal stem cells do not express CD10.

Aspect 46. The method of aspect 44, wherein said skeletal muscle mesenchymal stem cells do not express CD2, CD5, CD14, CD19, CD33, CD45, and DRII.

Aspect 47. The method of aspect 40, wherein said bone marrow mesenchymal stem cells express CD13,CD34, CD56, CD90, CD117 and nestin, and which do not express CD2, CD3, CD10, CD14, CD16, CD31, CD33, CD45 and CD64.

Aspect 48. The method of aspect 9, wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Aspect 49. The method of aspect 48, wherein said subepithelial umbilical cord derived mesenchymal stem cells do not express markers selected from a group comprising of; a)CD45; b) CD34; c) CD14; d) CD79; e) CD106; f) CD86; g) CD80; h) CD19; i) CD117; j) Stro-1 and k) HLA-DR.

Aspect 50. The method of aspect 48, wherein said subepithelial umbilical cord derived mesenchymal stem cells express CD29, CD73, CD90, CD166, SSEA4, CD9, CD44, CD146, and CD105.

Aspect 51. The method of aspect 48, wherein said subepithelial umbilical cord derived mesenchymal stem cells do not express CD45, CD34, CD14, CD79, CD106, CD86, CD80, CD19, CD117, Stro-1, and HLA-DR.

Aspect 52. The method of aspect 48, wherein said subepithelial umbilical cord derived mesenchymal stem cells are positive for SOX2.

Aspect 53. The method of aspect 48, wherein said subepithelial umbilical cord derived mesenchymal stem cells are positive for OCT4.

Aspect 54. The method of aspect 48, wherein said subepithelial umbilical cord derived mesenchymal stem cells are positive for OCT4 and SOX2.

Aspect 55. The method of aspect 1, wherein said mesenchymal stem cells are administered by an ommaya reservoir.

Aspect 56. The method of aspect 1, wherein said mesenchymal stem cells are administered intraventricularly.

Aspect 57. The method of aspect 1, wherein conditioned media from mesenchymal stem cells is administered instead of mesenchymal stem cells.

Aspect 58. The method of aspect 1, wherein exosomes from mesenchymal stem cells are administered instead of mesenchymal stem cells.

Aspect 59. The method of aspect 57, wherein said conditioned media and said mesenchymal stem cells are administered concurrently.

Aspect 60. The method of aspect 58, wherein said exosomes and said mesenchymal stem cells are administered concurrently.

### EXAMPLE

A patient suffering from lower back pain presented with a pain scale of 7 to 8 out of 10. The patient could not stand still for more than 15 seconds because he had intense pain. He could play golf but would have intense pain after playing which would not disappear. Had numbness in his toes and hands. Had knee and shoulder pain at all times. Could not walk more than 50 to 100 meters.

The patient underwent treatment from Jan. 18^{th}-29th 2016. Treatment comprised of IV, IT and IA infusions of hUC MSCs.

After treatment the patient could stand still on his feet for more than 5 minutes. He can play golf and experiences some pain after playing but after he goes to sleep, he wakes up in the morning without pain. Still has numbness in his toes and hands but less intense. Patient only has knee pain when he lies down and extends his knee completely (at night when he is in bed). Shoulder improved at the beginning but he now feels its worse (he is playing more golf now so it is probable that it's because of that). Patient has not tried walking for more than 100 mts at the time.

Before treatment: He used to take Lyrica, Oxycodone at all times due to extreme chronic pain. After treatment: The patient discontinued Lyrica and Oxycodone right after he got the treatment. He has NOT needed any of them to manage pain since then. He still takes Celebrex due to the knee and shoulder pain. People refer the patient looks better and healthy over all. The patient's pain score after treatment was 2 out of 10.

### REFERENCES

Rubin, D. I., Epidemiology and risk factors for spine pain. Neurol Clin, 2007. 25(2): p. 353-71.

Luo, X., et al., Estimates and patterns of direct health care expenditures among individuals with back pain in the United States. Spine (Phila Pa 1976), 2004. 29(1): p. 79-86.

Pengel, L. H., et al., Acute low back pain: systematic review of its prognosis. BMJ, 2003. 327(7410): p. 323.

Smith, C. and K. Grimmer-Somers, The treatment effect of exercise programmes for chronic low back pain. J Eval Clin Pract, 2010. 16(3): p. 484-91.

Woolf, A. D. and B. Pfleger, Burden of major musculoskeletal conditions. Bull World Health Organ, 2003. 81(9): p. 646-56.

Koes, B. W., M. W. van Tulder, and S. Thomas, Diagnosis and treatment of low back pain. BMJ, 2006. 332(7555): p. 1430-4.

Mehling, W. E., et al., Clinical decision rule for primary care patient with acute low back pain at risk of developing chronic pain. Spine J, 2015. 15(7): p. 1577-86.

Chou, R., et al., Diagnosis and treatment of low back pain: a joint clinical practice guideline from the American College of Physicians and the American Pain Society. Ann Intern Med, 2007. 147(7): p. 478-91.

van Tulder, M., et al., Chapter 3. European guidelines for the management of acute nonspecific low back pain in primary care. Eur Spine J, 2006. 15 Suppl 2: p. S169-91.

Roelofs, P. D., et al., Non-steroidal anti-inflammatory drugs for low back pain. Cochrane Database Syst Rev, 2008(1): p. CD000396.

Naesdal, J. and K. Brown, NSAID-associated adverse effects and acid control aids to prevent them: a review of current treatment options. Drug Saf, 2006. 29(2): p. 119-32.

Herrmann, W. A. and M. S. Geertsen, Efficacy and safety of lornoxicam compared with placebo and diclofenac in acute sciatica/lumbo-sciatica: an analysis from a randomised, double-blind, multicentre, parallel-group study. Int J Clin Pract, 2009. 63(11): p. 1613-21.

Van Pham, P., et al., Isolation and proliferation of umbilical cord tissue derived mesenchymal stem cells for clinical applications. Cell Tissue Bank, 2015.

Fazzina, R., et al., A new standardized clinical-grade protocol for banking human umbilical cord tissue cells. Transfusion, 2015. 55(12): p. 2864-73.

Bieback, K., Platelet lysate as replacement for fetal bovine serum in mesenchymal stromal cell cultures. Transfus Med Hemother, 2013. 40(5): p. 326-35.

Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.

Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.

Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.

Friedman, R., et al., Umbilical cord mesenchymal stem cells: adjuvants for human cell transplantation. Biol Blood Marrow Transplant, 2007. 13(12): p. 1477-86.

Hammadi, A. M., A. Marino, and S. Farhan, Clinical outcome of 50 progressive multiple sclerosis patients treated with cellular therapy in Iraq. Int J Stem Cells, 2011. 4(2): p. 113-5.

Kishk, N. A., et al., Case control series of intrathecal autologous bone marrow mesenchymal stem cell therapy for chronic spinal cord injury. Neurorehabil Neural Repair, 2010. 24(8): p. 702-8.

Yamout, B., et al., Bone marrow mesenchymal stem cell transplantation in patients with multiple sclerosis: a pilot study. J Neuroimmunol, 2010. 227(1-2): p. 185-9.

Karussis, D., et al., Safety and immunological effects of mesenchymal stem cell transplantation in patients with multiple sclerosis and amyotrophic lateral sclerosis. Arch Neurol, 2010. 67(10): p. 1187-94.

Dongmei, H., et al., Clinical analysis of the treatment of spinocerebellar ataxia and multiple system atrophy-cerebellar type with umbilical cord mesenchymal stromal cells. Cytotherapy, 2011. 13(8): p. 913-7.

Kim, H. Y., et al., Biological markers of mesenchymal stromal cells as predictors of response to autologous stem cell transplantation in patients with amyotrophic lateral sclerosis: an investigator-initiated trial and in vivo study. Stem Cells, 2014. 32(10): p. 2724-31.

Miao, X., X. Wu, and W. Shi, Umbilical cord mesenchymal stem cells in neurological disorders: A clinical study. Indian J Biochem Biophys, 2015. 52(2): p. 140-6.

Baek, W., et al., Stem cell transplantation into the intraventricular space via an Ommaya reservoir in a patient with amyotrophic lateral sclerosis. J Neurosurg Sci, 2012. 56(3): p. 261-3.

Clemons-Miller, A. R., et al., Intrathecal cytotoxic T-cell immunotherapy for metastatic leptomeningeal melanoma. Clin Cancer Res, 2001. 7(3 Suppl): p. 917s-924s.

Quattrocchi, K. B., et al., Pilot study of local autologous tumor infiltrating lymphocytes for the treatment of recurrent malignant gliomas. J Neurooncol, 1999. 45(2): p. 141-57.

Merchant, R. E., et al., Intralesional infusion of lymphokine-activated killer (LAK)cells and recombinant interleukin-2 (rIL-2)for the treatment of patients with malignant brain tumor. Neurosurgery, 1988. 23(6): p. 725-32.

Naganuma, H., et al., Complete remission of recurrent glioblastoma multiforme following local infusions of lymphokine activated killer cells. Case report. Acta Neurochir (Wien), 1989. 99(3-4): p. 157-60.

Yoshida, S., et al., Local administration of autologous lymphokine-i activated killer cells and recombinant interleukin 2 to patients with malignant brain tumors. Cancer Res, 1988. 48(17): p. 5011-6.

1. A method of reducing pain in a patient comprising administration of mesenchymal stem cells intrathecally in said patient.

2. The method of claim 1, wherein said pain is selected from a group comprising of: a) neuropathic pain; b) nociceptive pain; c) phantom pain; d) psychogenic pain; e) incident pain; f) breakthrough pain; g) discogenic pain and h) idiopathic pain.

3. The method of claim 1, wherein said mesenchymal stem cells are selected from a group of cells comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

4. The method of claim 3, wherein said mesenchymal stem cells express markers selected from a group comprising of: a) CD73; b) CD90; and c) CD105; and substantially lack expression of markers selected from a group comprising of: a) CD45; b) CD34; and c) CD14.

5. The method of claim 3, wherein said mesenchymal stem cells are capable of differentiating into one or more lineages selected from: a) adipocytic; b) chondrocytic; and c) osteocytic.

6. The method of claim 3, wherein said mesenchymal stem cells maintain a normal karyotype upon passaging for more than 20 cell doublings.

7. The method of claim 3, wherein said mesenchymal stem cells are capable of producing HGF-1 in vitro.

8. The method of claim 3, wherein said mesenchymal stem cells from umbilical cord tissue express markers selected from a group comprising of; a) oxidized low density lipoprotein receptor 1, b) chemokine receptor ligand 3; and c) granulocyte chemotactic protein.

9. The method of claim 8, wherein said mesenchymal stem cells from umbilical cord tissue do not express substantial markers selected from a group comprising of: a) CD117; b) CD31; c) CD34; and CD45;

10. The method of claim 9, wherein said mesenchymal stem cells from umbilical cord tissue express, relative to a human fibroblast, increased levels of interleukin 8 and reticulon 1

11. The method of claim 9, wherein said mesenchymal stem cells from umbilical cord tissue have the potential to differentiate into cells of at least a skeletal muscle, vascular smooth muscle, pericyte or vascular endothelium phenotype.

12. The method of claim 9, wherein said mesenchymal stem cells from umbilical cord tissue express markers selected from a group comprising of: a) CD10; b) CD13; c) CD44; d) CD73; and e) CD90.

13. The method of claim 9, wherein said umbilical cord tissue mesenchymal stem cell is an isolated umbilical cord tissue cell isolated from umbilical cord tissue substantially free of blood that is capable of self-renewal and expansion in culture,

14. The method of claim 9, wherein said umbilical cord tissue mesenchymal stem cells have the potential to differentiate into cells of other phenotypes.

15. The method of claim 9, wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1

16. The method of claim 1, wherein said mesenchymal stem cells are administered intraventricularly.

17. The method of claim 1, wherein conditioned media from mesenchymal stem cells is administered instead of mesenchymal stem cells.

18. The method of claim 1, wherein exosomes from mesenchymal stem cells are administered instead of mesenchymal stem cells.

19. The method of claim 1, wherein said conditioned media and said mesenchymal stem cells are administered concurrently.

20. The method of claim 1, wherein said exosomes and said mesenchymal stem cells are administered concurrently.
